(19) United States
(12) Patent Application Publication
(10) Pub. No.: US 2022/0257506 A1
(43) Pub. Date: Aug. 18, 2022

(54) Alkaline Drink

(71) Applicant: WET HOLDINGS (GLOBAL) LIMITED, Reading (GB)

(72) Inventors: Michael Adams, Knowle, Solihull (GB); Ahmed Mohamed, Birmingham (GB)

(21) Appl. No.: 17/610,866

(22) PCT Filed: May 15, 2020
(86) PCT No.: PCT/GB2020/000051

§ 371 Date: Nov. 12, 2021

(30) Foreign Application Priority Data
May 15, 2019 (GB) 1906865.9

(51) Int. Cl. A61K 9/00; A61K 31/366; A61K 31/22; A61K 31/40; A61K 31/47; A61K 31/4418; A61K 31/405; A61K 31/505; A61K 47/10; A61K 47/24; A61K 47/44; A61K 31/05; A61K 31/352; A61K 47/40; A61K 47/18

(52) U.S. Cl. CPC A61K 9/0095; A61K 47/183; A61K 31/22; A61K 31/40; A61K 31/47; A61K 31/4418; A61K 31/405; A61K 31/505; A61K 47/10; A61K 47/24; A61K 47/44; A61K 31/05; A61K 31/352; A61K 47/40; A61K 31/366

(57) ABSTRACT

A drink comprising in admixture stable alkaline water and a statin or a statin precursor, the pH of the water being from 8 to 10.5. The drink may include other ingredients including CBD or THC.

### FIELD OF THE INVENTION

The present invention relates to drinks for lowering or maintaining blood pressure.

### BACKGROUND OF THE INVENTION

Systemic arterial hypertension (also known as hypertension) is characterised by persistently high blood pressure (BP) in the systemic arteries. BP is commonly expressed as the ratio of the systolic BP (the pressure that the blood exerts on the arterial walls when the heart contracts) and the diastolic BP (the pressure when the heart relaxes). According to the European Society of Cardiology (ESC) and the European Society of Hypertension (ESH), hypertension is defined as office systolic BP (SBP) values ≥140 mmHg and/or diastolic BP (DBP) values 90 mmHg. The same classification is used in younger, middle-aged, and older people, whereas BP centiles are used in children and teenagers, for whom data from interventional trials are not available.

The global prevalence of hypertension was estimated to be 1.13 billion in 2015, with a prevalence of over 150 million in central and eastern Europe. The overall prevalence of hypertension in adults is around 30-45%, with a global age-standardized prevalence of 24% and 20% in men and women, respectively, in 2015. This high prevalence of hypertension is consistent across the world, irrespective of income status, i.e. in lower, middle, and higher income countries. Hypertension becomes progressively more common with advancing age, with a prevalence of >60% in people aged >60 years. As populations age, adopt more sedentary lifestyles, and increase their body weight, the prevalence of hypertension worldwide will continue to rise. It is estimated that the number of people with hypertension will increase by 15-20% by 2025, reaching close to 1.5 billion.

One of the most common methods for reduction of blood pressure is the use of statins. Statins are a group of medicines that can help lower the level of low-density lipoprotein (LDL) cholesterol in the blood. LDL cholesterol is often referred to as “bad cholesterol”, and statins reduce the production of it inside the liver. This is achieved by hepatic inhibition of cholesterol synthesis leading to an upregulation of LDL receptors which finally enhances the clearance of LDL from the serum. Statins also have a direct impact on the vascular system, which is potentially relevant for prevention of atherosclerotic complications. Some of these effects are mediated by isoprenoid intermediates involved in cholesterol biosynthesis, which regulates cellular distribution and function of small GTPases. Therefore, the inhibition of cholesterol synthesis with statin therapy may have an effect on adrenal and gonadal steroidogenesis because hormone synthesis requires an efficient intracellular pool of free cholesterol. A possible impairment of steroidogenesis could be due to the direct inhibition of cholesterol synthesis or could be caused by a reduction of LDL-particle uptake by steroidogenesis tissues.

A number of studies have assessed the impact of HMG-CoA reductase inhibitors on steroidogenesis. In vivo, dogs treated with very high doses of lovastatin showed a reduction in testicular endocrine function. In addition, simvastatin (20 mg/kg per day) lowered stimulated progesterone levels in rabbits with defective LDL-receptors. However, in humans given up to 80 mg/day lovastatin, no effect on adrenal and gonadal steroidogenesis was demonstrated under basal conditions. This is further supported by studies demonstrating no clear adverse effect on basal gonadotropin and sex hormone serum levels in hypercholesterolemic patients given statins. There are currently 2 main types of statins: Type I statins, Type II statins. Type I statins have a substituted decalin-ring and include pravastatin, lovastatin, mevastatin, and simvastatin. Type II statins typically have a fluorophenyl group in place of the butyrl group that is present in Type I statins. Exemplary Type II statins include atorvastatin, fluvastatin, rosuvastatin, cerivastatin, and pitavastatin. Statins may also be present in formulations in combinations of more than one form, i.e. a statin may be present as an acid (e.g. carboxylic acid), salt (including calcium, sodium, potassium, and magnesium salts), or neutral (closed lactone ring) form. The dosage and administration of statins are now regulated by strict medial guidelines, such as the guidelines of the National Institute of for Health and Care Excellence (NICE) and the European Society of Cardiology's guidelines. In brief, each type of statin has lowest acceptable and highest permissible doses.

Another approach for managing BP levels is the modulation of blood viscosity. The relationship between BP and viscosity is such that, given a constant systolic BP, if blood viscosity increases, then the total peripheral resistance (TPR) will necessarily increase, thereby reducing blood flow. Conversely, when viscosity decreases, blood flow and perfusion will increase. Because of the dependence of systemic arterial BP on cardiac output and TPR, if blood viscosity and TPR rise, systolic BP must then increase for cardiac output to be maintained. Consequently, blood viscosity has been established as a major determinant of the work of the heart and tissue perfusion. Since increased viscosity requires a higher BP to ensure the same circulating volume of blood, both the burden on the heart and the forces acting on the vessel wall are directly modulated by changes in blood viscosity.

Three important studies helped establish the relationship between blood pressure and blood viscosity. The earliest study observed 49 normal subjects and 49 patients with untreated essential hypertension, showing a direct correlation between BP and blood viscosity among both normotensive and hypertensive subjects (p<0.001). Systolic blood viscosity was 8 to 10% higher in hypertensive patients compared with normotensive controls, and diastolic blood viscosity was 16 to 28% higher in hypertensive patients. Subgroups each comprised of 25 subjects having matched hematocrits were also compared, and viscosity remained significantly higher in hypertensive subjects (p<0.05).

In the Edinburgh Artery Study, which followed 1,592 randomly selected adults, demonstrated that systolic BP was univariately related to blood viscosity in males only (p<0.001), and diastolic BP was univariately related to blood viscosity in both sexes (p<0.001). Hematocrit-corrected blood viscosity levels were also significantly related to systolic and diastolic BP in both sexes. The study's authors suggested that the strong, independent relationship between viscosity and BP cannot be explained by hematocrit and plasma content alone, but erythrocyte deformability and fibrinogen made contributions. Moreover, they wrote that the pathophysiological significance of blood viscosity in hypertension related to its modulation of TPR.

A third study followed 331 males newly diagnosed with essential hypertension for up to 12 years. The researchers grouped patients into three categories by their diastolic blood viscosity levels: high, medium and low. The highest tertile for diastolic viscosity were more than three times as likely to have cardiovascular events than the lowest diastolic viscosity tertile (hazard ratio=3.42, 95% confidence interval=1.40-8.38, p=0.006). The third study's authors concluded that both blood viscosity and hematocrit were univariate predictors of cardiovascular morbidity in hypertensive men but only viscosity came out as an independent risk factor in a multivariate analysis, thus supporting the perspective that diastolic blood viscosity “as a global marker of the whole-blood rheological properties may be a better discriminant of cardiovascular risk in hypertensive men.” They added that blood viscosity, an overall measure of flow resistance of bulk blood, depends on several factors, including cell concentration, cell aggregation, cell deformability and plasma protein concentration.

Blood viscosity holds certain similarities with blood pressure. Like blood pressure, the viscosity of blood changes during each cardiac cycle and is reported using two numerical quantities: systolic and diastolic viscosity. However, while blood pressure is a parameter of the circulatory system as a whole, blood viscosity is a parameter specific to the fluid flowing through the system. Therefore, viscosity can be said to precede pressure and to be biophysically more fundamental than pressure.

Cannabidiol (CBD) can induce blood pressure lowering effects on consumers. CBD is a phytocannabinoid discovered in 1940. It is one of some 113 identified cannabinoids in cannabis plants and accounts for up to 40% of the plant's extract. CBD is an essential component of medical marijuana, it is derived directly from the hemp plant, which is a cousin of the marijuana plant. While CBD is a component of marijuana, by itself it does not cause psychotropic effects. According to a report from the World Health Organization, “In humans, CBD exhibits no effects indicative of any abuse or dependence potential. . . . To date, there is no evidence of public health related problems associated with the use of pure CBD.”

CBD can induce numerous cardiovascular benefits in preclinical research, including a reduced blood pressure (BP) response to stress. Additionally, in a randomized, placebo-controlled, double-blind, crossover study, nine healthy male volunteers were given 600 mg of CBD or placebo. Cardiovascular parameters were monitored using a finometer and laser Doppler. The results varied but participants had a decrease of about 6 mm Hg. The data show that a single dose of CBD reduces resting blood pressure and the blood pressure response to stress, particularly cold stress, and especially in the post-test periods. This may reflect the anxiolytic and analgesic effects of CBD, as well as any potential direct cardiovascular effects. In fact, epidemiological studies have shown a positive relationship between long-term stress and the development of cardiovascular disease. Factors like social isolation, low socioeconomic status, depression, stressful family and work life, and anxiety are associated with an increased risk of the development and accelerated the progression of existing cardiovascular disease.

Tetrahydrocannabinol (THC) is also one of the cannabinoids identified in cannabis. THC 5 is the principal psychoactive constituent of cannabis. With chemical name (−)-trans-Δ⁹-tetrahydrocannabinol, the term THC also refers to cannabinoid isomers. THC, along with its double bond isomers and their stereoisomers, is one of only three cannabinoids scheduled by the UN Convention on Psychotropic Substances (the other two are dimethylheptylpyran and parahexyl). It was listed under Schedule I in 1971, but reclassified to Schedule II in 1991 following a recommendation from the WHO. Based on subsequent studies, the WHO has recommended the reclassification to the less stringent Schedule III. As of October 2018 when recreational use of cannabis was legalized in Canada, some 220 dietary supplements and 19 veterinary health products containing not more than 10 parts per million of THC extract were approved with general health claims for treating minor conditions.

Among the health benefits of THC, a systematic review into the current evidence on the associations between THC and cardiovascular diseases found that acute THC dosing reduced BP and heart rate in anaesthetised animals, conscious animals and animal models of 20 stress or hypertension and increased cerebral BF in murine stroke models. Chronic dosing increased BF in large arteries in anaesthetised animals and reduced BP in models of stress or hypertension. In humans, acute administration increased HR. THC acts differently according to species and experimental conditions, causing bradycardia, hypotension and increased BF in animals; and causing increased HR in humans.

### STATEMENTS OF THE INVENTION

In this present invention, statin(s) and, optionally, other ingredients are added to stable alkaline water to produce a stable drink with the aim of reducing or maintaining the blood pressure of consumers.

Preferably, CBD and/or THC are added to the statin containing alkaline drink formulations in this invention to augment the blood pressure lowering effects and to reduce consumers' anxiety which is considered as a key contributory factor for high blood pressure.

Specifically, the invention provides drink formulations containing stable alkaline water (pH 8-10,5, preferably 9-10.5), at least one statin agent and at least one solubiliser. These drink formulations are useful for lowering blood viscosity, lowering total cholesterol and the management of diseases that are associated with high cholesterol, such as hypertension, Heterozygous Familial Hypercholesterolemia (HeFH) and cardiovascular diseases. These drinks can also be useful for managing children, adolescents, and other individuals to whom tablets, or capsule formulations are difficult or impractical to administer. A preferred drink formulation includes simvastatin.

The invention provides drink formulations comprising stable alkaline water produced by nonmagnetic suspended agitation process (n-MSAP) by using the Activated Enhancement System (AES) which provides stable alkaline water with pH ranging from 8-10.5. Reference is made to our copending patent application WO 2019/243759. Unlike alkaline water produced by electrolysis or ionisation, the alkaline water produced by the method and apparatuses included in that patent produce stable alkaline water that does not lose its alkaline pH for extended periods of times.

Accordingly, the preferred method of making the alkaline water makes use of apparatus comprising a vessel having a water inlet and a water outlet, means for feeding water to the vessel via the water inlet, the vessel containing a body of water and a solid particulate or granular material comprising one or more elementary metals or oxides thereof capable of raising the pH of the water, and means, located within the vessel and connected to the water inlet, for causing circulatory motion of water entering the vessel sufficient to suspend the solid material within the body of water during passage of water through the vessel.

Preferably, the means for causing circulatory motion increases the flow rate of water entering the vessel. More preferably, the means for causing circulatory motion comprises a venturi effect inducing device.

In this invention, the drink formulations comprise the latter alkaline water with 0.01-25 mg/ml of statin, preferably formulations will include 1-5 mg/ml of statin, more preferably formulations include about 2 mg/ml of statin. The total amount of statin in a formulation may be due to a single statin or a combination of statins. Combinations of statins may, for example, include one or more Type I statins, Type II statins, or combinations thereof.

Herein, Type I statins have a substituted decalin-ring and include pravastatin, lovastatin, mevastatin, and simvastatin. Type II statins typically have a fluorophenyl group in place of the butyrl group that is present in Type I statins. Exemplary Type II statins include atorvastatin, fluvastatin, rosuvastatin, cerivastatin, and pitavastatin.

Statins may also be present in the formulations in combinations of more than one form, i.e. a statin may be present as an acid (e.g. carboxylic acid), salt (including calcium, sodium, potassium, and magnesium salts), or neutral (closed lactone ring) form. Those of skill in the art will recognize that the desired combination of statins will depend upon the proposed end user, the objective of the treatment, and the solubility of the combination.

Formulations of the invention may also include a vehicle to solubilize the statin (i.e. a solubilizer or solubilizing agent). It is envisioned that liquid formulations of the invention may include one or more of the following vehicles: stable alkaline water, propylene glycol, minerals, propylene glycolmonostearate, propylene glycol alginate, natural glycerine, niacin, synthetic glycerine, vitamins, sorbitol, alcohols, myristyl alcohol, carboxymethylcellulose, labrasol, copovidone, Captex 355, croscarmellose sodium, polyethylene glycol (PEG) 400, PEG 1000, PEG 1450, PEG 1540, crospovidone, ethyl cellulose, aqueous polysorbate 20, aqueous polysorbate 40, aqueous polysorbate 60, aqueous polysorbate 80, cellulose, oxidized cellulose, polyoxyl 10 oleoyl ether, cellulose sodium phosphate, polyoxyl 20 cetostearyl, hyopromellose, poloyxyl 35 castor oil,

polyoxyl 40 hydrogentated castor oil, polyoxyl 40 stearate, poloxyl lauryl ether, poloxyl oleate, poloxyl stearyl ether, or any combination thereof.

Preferred embodiments of drink formulations of the invention may also include an antioxidant, flavoring, preservative, or a combination thereof. More preferably, oral solutions include all three elements (i.e. an antioxidant, a flavoring, and a preservative). Preferred antioxidants may include butylated hydroxytoluene (BHT), butylated hydroxyanisole (BHA), and combinations thereof.

Suitable preservatives comprise methylparaben, methylparaben sodium, propylparaben, and combinations thereof. Propylparaben and methylparaben are preferred preservatives, and combinations of the two are more preferred.

Flavorings suitable to include in drinks formulations of the invention are natural flavours including, but not limited to orange, red fruits, lemon lime, vanilla, apple, and various combinations thereof.

Other elements that optionally may be included in formulations of the invention include amino acids, vitamins, minerals, phospholipids, cyclodextrins, triglycerides, diglycerides, monoglycerides, ionic surfactants, non-ionic surfactants, fatty acids, buffers, or any combinations thereof.

Phospholipids suitable for inclusion in formulations of the invention may include phosphotidyl choline, phophotidyl ethanolamine, sphingomylein, lauroyl polyoxylglyceride, linoleoyl polyoxylglyceride, oleoyl polyoxylglyceride, lecithin, soy isoflavones, and combinations thereof.

Cyclodextrins suitable for inclusion in liquid solutions may include α cyclodextrin, β cyclodextrin, δ cyclodextrin, γ cyclodextrin, and combinations thereof.

Suitable triglycerides that may be included in formulations of the invention are olive oil, safflower oil, soybean oil, sunflower oil, or combinations thereof. Fatty acids that may be included in the invention are oleic acid, stearic acid, α-lipoic acid, ethyl oleate, myristic acid, palmitic acid, sorbitan monolaurate, sorbitan monooleate, sorbitan monopalmitate, sorbitan monostearate, sorbitan trioleate, and combinations thereof.

Any of the following amino acids may be included in formulations of the invention: alanine, arginine, aspartic acid, choline, folic acid, glutamine, glycine, histidine, isoleucine, leucine, lysine, methionine, phenylalanine, serine, threonine, tryptophan, tyrosine, valine, and combinations thereof.

Buffers may be included in formulations of the invention. In particular, any of the following may be included: ascorbic acid, ascorbyl palmitate, calcium sulfate, citric acid, dibasic sodium phosphate, monobasic sodium phosphate, potassium carbonate, potassium citrate, sodium acetate, sodium ascorbate, sodium bicarbonate, sodium carbonate, sodium citrate, sodium lauryl sulfate, sodium metabisulfate, and combinations thereof.

Surfactants that may be suitable for inclusion in formulations are aqueous sodium laurel sulfate, a tocopherol excipient, tocopherol polyethyleneglycol, beta-carotene, lycopene, and combinations thereof.

Preferably, CBD and/or THC can be added to the alkaline formulations. Suitable CBD for inclusion in formulations are CBD oil or CBD water soluble powder with concentrations preferably ranging from 2 mg to 600 mg or more. On the other hand, THC is added only by using THC containing oils and at concentrations up to 10 ppm.

While the addition of CBD water soluble powder or paste can be easily achieved by using ultrasonic mixing or any other powder-in-water mixing and homogenizing techniques, the addition of CBD oil and/or THC containing oils to the formulations can be achieved by high-energy approaches and low-energy approaches. Suitable high-energy approaches include a) high-pressure valve homogenizers or b) microfluildizers or c) ultrasonic techniques. On the other hand, low-energy approaches include a) phase inversion methods or b) spontaneous emulsification method.

It will be clear to the skilled artisan that a variety of optional ingredients may be included in formulations of the invention. An exemplary embodiment of the invention comprises drink formulations comprising stable alkaline water and weight to weight (w/w) 0.2% simvastatin or simvastatin and atorvastatin, 2 mg-600 mg CBD, up to 10 ppm THC, polyethylene glycol, USP, methylparaben, NF, propylparaben, NF, butylated hydroxytoluene, NF, glycerine, USP, 0.1% a first flavoring, 0.15% a second flavoring, and 0.1% a third flavoring. Preferred flavorings are natural flavours of vanilla or apple, or lemon lime, or red fruits or orange or any other natural flavors.

Several preferred embodiments of formulations include, but are not limited to, the following: 0.2% (w/w) simvastatin or simvastatin and atorvastatin, 59.8% polyethylene 25 glycol (PEG) 400, 0.2% methylparaben, 0.02% propylparaben, 0.01% butylated hydroxyanisole (BHA), 0.1% lemon lime flavor, 2 mg-600 mg CBD, 10 ppm THC, and stable alkaline water.

Ideally, dosages are monitored regularly using techniques well-known to the skilled artisan and adjusted as needed, preferably about every four weeks, to achieve the desired goal(s).

Herein, “individual” or “subject” refers to a human unless otherwise specified. Humans include children and adolescents.

“Statin” or “statins” is used herein to generally refer to a class of drugs that lower cholesterol levels by inhibiting the enzyme HMG-CoA reductase.

Unless specified, it is understood that “statin” refers to both a single composition or a combination of compositions.

Unless defined otherwise, all technical and scientific terms used herein have the same meaning as is commonly understood by one of skill in the art to which this invention belongs at the time of filing.

### DETAILED DESCRIPTION OF THE INVENTION

This invention provides formulations that provide stable drinks comprising one or more statins, e.g. simvastatin, atorvastatin, etc., as the active ingredient(s). The drinks are suitable for oral ingestion and can help reduce high cholesterol and particularly suitable to manage hypertension, familial hypercholesterolemia, particularly heterozygous familial hypercholesterolemia (HeFH) and other cardiovascular diseases. Preferably the invention is used as an adjunct to diet and lifestyle modifications to reduce total cholesterol, low density lipoprotein cholesterol (LDL-C), and Apolipoprotein B (Apo B) levels.

In this invention, drink formulations include a stable alkaline water which is produced by the Activated Enhancement System (AES) which utilises a non-magnetic suspended agitation process (n-MSAP) to produce stable alkaline water with pH levels ranging from 8-10.5. This method enables the production of alkaline water that can stably hold active ingredients, including statin(s). Also, this method enables production of alkaline water that can hold electrolytes, vitamins, flavourings, minerals, proteins and other nutritional elements. Reference is made to our patent application No WO 2019/243759.

According to research, alkaline water hydrates the body more efficiently than neutral water as it reduces the blood viscosity of the consumer. This is particularly beneficial in patients with hypertension and similar cardiovascular diseases because it will reduce the pressure on the blood vessels. In addition to the alkaline water and statin(s), the drink formulations in this invention may comprise a solubilizer that allows the statin(s) to enter and remain in solution.

Potential solubilizers may include: alkaline water, propylene glycol, minerals, propylene glycolmonostearate, propylene glycol alginate, natural glycerine, niacin, synthetic glycerine, vitamins, sorbitol, alcohols, myristyl alcohol, carboxymethylcellulose, labrasol, copovidone, Captex 355, croscarmellose sodium, polyethylene glycol (PEG) 400, other PEGs (e.g. 200, 300, 1000, 1450, 1540, etc.), crospovidone, ethyl cellulose, aqueous polysorbate 80, cellulose, other polysorbates (20, 40, 60), oxidized cellulose, polyoxyl 10 oleoyl ether, cellulose sodium phosphate, polyoxyl 20 cetostearyl, hyopromellose, poloyxyl 35 castor oil, polyoxyl 40 hydrogentated castor oil, polyoxyl 40 stearate, poloxyl lauryl ether, poloxyl oleate, and poloxyl stearyl ether.

Exemplary solutions of the invention may include stable alkaline water, statin, such as simvastatin, at least one vehicle to solubilize the statin, and optionally, one or more antioxidants, flavors, or preservatives. Vitamins, amino acids, minerals, phospholipids, cyclodextrins, triglycerides, diglycerides, monoglycerides, surfactants, fatty acids, or buffers also may be included in the formulations of the invention.

It is envisioned that formulations of the invention may include any statin that is not presently available in a liquid formulation that can be ingested orally and can be solubilized using one or more of the solublizers disclosed herein by using the methods disclosed herein. Combinations of statins may be used and include one or more Type I or Type II statins or combinations thereof. Suitable Type I statins include lovastatin, mevastatin, pravastatin, and simvastatin. Suitable Type II statins typically have a fluorophenyl group in place of the butyrl group that is present in Type I statins and include atorvastatin, cerivastatin, fluvastatin, rosuvastatin, and pitavastatin.

Multiple forms of statins may also be used in the formulations. That is, a statin may be present as an acid (e.g. carboxylic acid), salt (including calcium, sodium, potassium, and magnesium salts), or neutral (closed lactone ring) form. Those of skill in the art will recognize that the desired combination(s) of statins will depend upon the end user, the objective(s) of the treatment, and the solubility of the combination.

Phospholipids suitable for inclusion in oral solutions or suspensions may include: phosphotidyl choline, phophotidyl ethanolamine, sphingomylein, lauroyl polyoxylglyceride, linoleoyl polyoxylglyceride, oleoyl polyoxylglyceride, lecithin, and soy isoflavones.

Solutions of the invention may also include α, β, δ, and γ cyclodextrins. Triglycerides may also be included in solutions of the invention, such as: olive oil, safflower oil, soybean oil, sunflower oil, diglycerides, monoglycerides, and diacetylated monoglycerides.

Fatty acids suitable for inclusion in solutions of the invention may include: oleic acid, stearic acid, α-lipoic acid, ethyl oleate, myristic acid, palmitic acid, sorbitan monolaurate, sorbitan monooleate, sorbitan monopalmitate, sorbitan monostearate, and sorbitan trioleate.

Amino acids suitable for inclusion in solutions of the invention may include:

alanine, arginine, aspartic acid, choline, folic acid, glutamine, glycine, histidine, isoleucine, leucine, lysine, methionine, phenylalanine, serine, threonine, tryptophan, tyrosine, and valine.

Suitable flavors may include: grape syrup, grape cherry syrup, bubble gum, almond oil, anise oil, cherry syrup, clove oil, lemon oil, licorice fluid extract, orange oil or syrup, peppermint oil, and vanilla tincture.

Possible buffers that may be included in the liquid solutions are: ascorbyl palmitate, calcium sulfate, citric acid, dibasic sodium phosphate, monobasic sodium phosphate, potassium carbonate, potassium citrate, sodium acetate, sodium ascorbate, sodium bicarbonate, sodium carbonate, sodium citrate, sodium lauryl sulfate, and sodium metabisulfate.

Preservatives that may be included in the invention are methylparaben, and methylparaben sodium. Antioxidants that may be included in the invention are butylated hydroxytoluene, and butylated hydroxyanisole. Any of the following ionic or non-ionic surfactants may be included in oral solutions or suspensions of the invention: sodium laurel sulfate in water, tocopherols excipient, tocopherol polyethyleneglycol, betacarotene, and lycopene.

Preferably, CBD and/or THC can be added to the drink formulations. Suitable CBD for inclusion in formulations are CBD oil or CBD water soluble powder with concentrations ranging from 2 mg to 600 mg or more. On the other hand, THC is added only by using THC containing oils and at concentrations up to 10 ppm.

Exemplary formulations are comprised of percent weight to weight (% w/w) of 0.05-10% simvastatin, and any of the following: 5-75% PEG 400, 5-75% propylene glycol, 5-75% glycerine, 5-60% chremophor EL, 5-60% labrasol (i.e. caprylocarproyl polyoxyglycerides), 5-60% Captex 355 (caprylic and capric acid triglycerides), 5-60% labrafil M 2125 CS (linoleoyl polyoxylglycerides), 5-60% Captex 500 P (glyceryl triacetate), 0.1-60% polysorbate 80, 0.1-60% 5% sodium lauryl sulfate in water, 0.01-1.5% methylparapben, 0.01-1.0% propylparaben, 1-15% sorbitol, 0.01-1.5% sodium benzoate, 0.01-0.5% sodium metabisulfate, 0.005-2.5% citric acid, 0-0.5% flavoring (e.g. bubble gum, grape, cherry), 0-2% saccharin sodium, 0-0.02% BHA, 0-0.02% BHT, 2 mg-600 mg CBD, up to 10 ppm THC, and stable alkaline water.

1. A drink comprising in admixture stable alkaline water and a statin or a statin precursor, the pH of the water being from 8 to 10.5.

2. The drink of claim 1, wherein the water is made using apparatus comprising a vessel having a water inlet and a water outlet, means for feeding water to the vessel via the water inlet, the vessel containing a body of water and a solid particulate or granular material comprising one or more elementary metals or oxides thereof capable of raising the pH of the water, and means, located within the vessel and connected to the water inlet, for causing circulatory motion of water entering the vessel sufficient to suspend the solid material within the body of water during passage of water through the vessel.

3. The drink of claim 2, wherein the means for causing circulatory motion increases the flow rate of water entering the vessel.

4. The drink of claim 2, wherein the means for causing circulatory motion comprises a venturi effect inducing device.

5. The drink of claim 1, wherein the statin is present in an amount of from 0.05 to 10% w/w.

6. The drink of claim 1, wherein the drink further comprises a solubiliser selected from one or more of propylene glycol, minerals, propylene glycolmonostearate, propylene glycol alginate, natural glycerine, niacin, synthetic glycerine, vitamins, sorbitol, alcohols, myristyl alcohol, carboxymethylcellulose, labrasol, copovidone, Captex 355, croscarmellose sodium, polyethylene glycol (PEG) 400, PEG 1000, PEG 1450, PEG 1540, crospovidone, ethyl cellulose, aqueous polysorbate 20, aqueous polysorbate 40, aqueous polysorbate 60, aqueous polysorbate 80, cellulose, oxidized cellulose, polyoxyl 10 oleoyl ether, cellulose sodium phosphate, polyoxyl 20 cetostearyl, hyopromellose, poloyxyl 35 castor oil, polyoxyl 40 hydrogentated castor oil, polyoxyl 40 stearate, poloxyl lauryl ether, poloxyl oleate and poloxyl stearyl ether.

7. The drink of claim 1, wherein the statin is selected from one or more of simvastatin, lovastatin, pravastatin, atorvastatin, pitavastatin, mevastatin, cerivastatin, fluvastatin and rosuvastatin.

8. The drink of claim 1, further comprising one or more of an antioxidant, a flavoring and a preservative.

9. The drink of claim 1, further comprising one or more of an amino acid, a vitamin, a mineral, a phospholipid, a cyclodextrin, a triglyceride, a diglyceride, a monoglyceride, a surfactant, a bile salt, a fatty acid, a sweetener and a buffer.

10. The drink of any claim 1, wherein the drink comprises an antioxidant selected from one or more of butylated hydroxytoluene (BHT) and butylated hydroxyanisole (BHA).

11. The drink of claim 1, wherein the drink comprises a flavoring selected from one or more of grape syrup, grape cherry syrup, bubble gum, almond oil, anise oil, cherry syrup, clove oil, lemon oil, liquorice fluid extract, orange oil, orange syrup, peppermint oil and vanilla tincture.

12. The drink of claim 1, wherein the drink comprises a preservative selected from one or more of propylparaben, methylparaben and methylparaben sodium.

13. The drink of claim 1, wherein the drink comprises a phospholipid selected from one or more of phosphotidyl choline, phophotidyl ethanolamine, sphingomylein, lauroyl polyoxylglyceride, linoleoyl polyoxylglyceride, oleoyl polyoxylglyceride, lecithin and soy isoflavones.

14. The drink of claim 1, wherein the drink comprises a cyclodextrin selected from one or more of an α cyclodextrin, β cyclodextrin, δ cyclodextrin and a γ cyclodextrin.

15. The drink of claim 1, wherein the drink comprises a triglyceride selected from one or more of olive oil, safflower oil, soybean oil and sunflower oil.

16. The drink of claim 1, wherein the drink comprises a fatty acid selected from one or more of oleic acid, stearic acid, α-lipoic acid, ethyl oleate, myristic acid, palmitic acid, sorbitan monolaurate, sorbitan monooleate, sorbitan monopalmitate, sorbitan monostearate and sorbitan trioleate.

17. The drink of claim 1, wherein the drink comprises an amino acid selected from one or more of alanine, arginine, aspartic acid, choline, folic acid, glutamine, glycine, histidine, isoleucine, leucine, lysine, methionine, phenylalanine, serine, threonine, tryptophan, tyrosine and valine.

18. The drink of claim 1, wherein the drink comprises any one or more of:
a) a buffer selected from one or more of ascorbyl palmitate, calcium sulfate, citric acid, dibasic sodium phosphate, monobasic sodium phosphate, potassium carbonate, potassium citrate, sodium acetate, sodium ascorbate, sodium bicarbonate, sodium carbonate, sodium citrate, sodium lauryl sulfate and sodium metabisulfate;
b) a surfactant selected from one or more of aqueous sodium laurel sulfate, a tocopherol excipient, tocopherol polyethyleneglycol, beta-carotene and lycopene;
c) CBD which is present as the result of the addition of CBD oil or CBD water soluble powder or paste; and
d) THC which is present as a result of the addition of a THC containing oil.

19. (canceled)

20. (canceled)

21. (canceled)

22. The drink of claim 1, wherein the pH of the water is from 9 to 10.5

23. A method of reducing or maintaining blood pressure comprising assimilating a drink according to claim 1.
